(19) United States
(12) Patent Application Publication
(10) Pub. No.: US 2024/0042229 A1
(43) Pub. Date: Feb. 8, 2024

(54) BIOLOGICAL OR NATURAL FREQUENCY STIMULATION

(71) Applicants: Haim Amir, Ramit-HaSharon (IL); Ohad Amir, Ohad (IL); Jonathan Mark Schnapp, Tel Aviv - Jaffa (IL)

(72) Inventors: Haim Amir, Ramit-HaSharon (IL); Ohad Amir, Ohad (IL); Jonathan Mark Schnapp, Tel Aviv - Jaffa (IL)

(21) Appl. No.: 18/382,181

(22) Filed: Oct. 20, 2023

Related U.S. Application Data
(63) Continuation-in-part of application No. PCT/IL22/50405, filed on Apr. 19, 2022.
(60) Provisional application No. 63/178,209, filed on Apr. 22, 2021.

(51) Int. Cl. A61N 5/06

(52) U.S. Cl. CPC A61N 5/06; A61N 2005/0658

(57) ABSTRACT

Systems and methods are disclosed to provide a modulated stimulation signal to a patient, the modulated stimulation signal including a carrier wave modulated with a biological or natural frequency stimulation signal, to provide the benefit of the biological or natural frequency stimulation signal to the patient without the patient perceiving the biological or natural frequency stimulation signal.

### CLAIM OF PRIORITY

This application is a continuation-in-part of PCT/IL2022/050405 filed on 19 Apr. 2022, and published as WO 2022/224251 on 27 Oct. 2022, which claims the benefit of priority of U.S. Patent Application No. 63/178,209 filed on 22 Apr. 2021, each of which are incorporated herein by reference in their entireties.

### TECHNICAL FIELD

This document relates generally to stimulation of the human body, and more particularly, but not by way of limitation, to biological or natural frequency stimulation to the human body.

### BACKGROUND

Human bodies are composed of trillions of individual cells of many different types performing different biological functions. Chemical reactions and ionic movements in relation to cells may create electrical and magnetic potentials and fields. Different organs and groups of cells and tissues create different potentials and fields, and different cells have different membrane potentials required for transport. Specific types of brain and cellular activity produce different fields in the body. For example, alpha waves are neural oscillations ranging in frequency and amplitude typically associated with a relaxed mental state. Alpha waves are generally defined as being between 7 and 14 Hz, and in certain examples, more narrowly 8 to 12 Hz or 8 to 13 Hz by some definitions. Different fields can be applied to the patient to provide different affects.

### SUMMARY

Systems and methods are disclosed to provide a modulated stimulation signal, the modulated stimulation signal including a carrier wave modulated with a biological or natural frequency stimulation signal, and to provide the modulated stimulation signal to a patient. In certain examples, the modulated stimulation signal can be configured to provide the benefit of the biological or natural frequency stimulation signal to the patient, such as to induce alpha wave or other neurological activity in the patient, without the patient perceiving the biological or natural frequency stimulation signal. In certain examples, the modulated stimulation signal can be configured to affect a mood of the patient, such as to induce or increase a pleasant or deactivated state or feeling of calm, relaxed, serene, or content. In other examples, the modulated stimulation signal can be configured to induce sleepiness or improve sleep quality or provide one or more other benefits to the patient. In some examples, the modulated stimulation signal can be configured to aid concentration or processing of new information.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example system for providing a modulated stimulation signal to a patient.

FIG. 2 illustrates example therapy sources.

FIGS. 3-4 illustrate example alpha waves of an electroencephalogram (EEG) signal.

FIG. 5 illustrates an example natural frequency signal.

FIGS. 6-7 illustrate example signal modulation.

FIG. 8 illustrates an example method of inducing alpha-wave activity in a patient.

FIG. 9 illustrates a block diagram of an example machine upon which any one or more of the techniques (e.g., methodologies) discussed herein may perform.

FIG. 10 illustrates an example layout of a printed circuit board (PCB).

FIG. 11 illustrates example measurements.

### DETAILED DESCRIPTION

The present inventors have recognized, among other things, that a carrier wave can be modulated with a biological or natural frequency stimulation signal to produce a modulated stimulation signal, and that the modulated stimulation signal can be provided to a patient to provide the benefit of the biological or natural frequency stimulation signal to the patient without the patient perceiving the biological or natural frequency stimulation signal.

In an example, the carrier wave can include a human-perceptible signal, such as a visible light or audible sound, or one or more other perceptible signals apparent to one or more of the five senses of the patient, including touch, taste, feel. In such examples, the modulated stimulation signal can be perceptible to the patient, but the biological or natural frequency stimulation signal may not be perceptible separately from the carrier wave, and accordingly, the modulated stimulation signal. In other examples, the carrier wave can include a human-imperceptible signal, such as a human-imperceptible carrier wave having a frequency outside the level of conscious human perception, in certain examples, provided separate from or in addition to a human-perceptible signal. In other examples, the carrier wave and the biological or natural frequency stimulation signal can each be human-imperceptible, as well as the combination, such that the modulated stimulation signal remans human-imperceptible. Similarly, depending on the carrier wave and a therapy source, the biological or natural frequency stimulation signal can itself be human perceptible or human imperceptible separate from the carrier wave, but when modulated onto the carrier wave, can be human imperceptible.

The range of human perception, what is perceptible to a human and what is not perceptible, varies with specific senses. Each of the five senses has a different range of conscious human perception. The range of audible human perception is generally understood as 20 Hz to 20 kHz. Under ideal laboratory conditions, humans can hear sound as low as 12 Hz and as high as 28 kHz, though the threshold increases sharply at 15 kHz in adults, corresponding to the last auditory channel of the cochlea. The human auditory system is most sensitive to frequencies between 2 kHz and 5 kHz. In contrast, the upper range of human visual perception, the frequency at which intermittent light stimulus is perceived as steady or constant to a human viewer, is generally understood as 60 to 90 Hz. Flicker is a temporal light artifact characterized by a rapid variation in light source intensity. Most humans do not perceive any changes in light (e.g., flicker, etc.) above 400 Hz. However, a human eye typically responds to signals having wavelengths between 380 and 750 nanometers, corresponding to a human perceptible frequency range of 400 THz and 790 THz. Accordingly, the stimulation signal can include changes in visible light at a frequency above that which is visible as flicker (e.g., above 90 Hz, 100 Hz, 400 Hz, etc.), but below that which becomes visible as light itself (e.g., 400 THz, etc.). For example, an electromagnetic wave having a frequency corresponding to visible light may be a carrier wave that undergoes amplitude modulation or amplitude key shifting (ASK) modulation with a low frequency signal so as to cause the flickering of the electromagnetic wave.

The carrier wave can be provided by a therapy source. In an example, the therapy source can include an audio or visual entertainment or convenience device, such as a room or accent light, a display screen, a projector, etc., configured to provide a human-perceptible carrier wave that is perceptible to the patient. The carrier wave can include a human-perceptible audio or visual signal from the therapy source. The human-perceptible carrier wave can be modulated with a biological or natural frequency stimulation signal to provide the modulated stimulation signal, such that the expected or requested audio or visual signal is perceptible to the patient, but the biological or natural frequency stimulation signal is not perceptible to the patient.

In other examples, the therapy source can include an electronic device configured to provide a human-imperceptible carrier wave that is not perceptible to the patient, including one or more of a light, audio, electric (e.g., applied electrical current using electrodes placed in contact with the skin, etc.), or electromagnetic source (e.g., one or more fixed or moving electromagnets, rotating permanent magnets, radio frequency (RF) source, electromagnetic field (EMF) or pulsed EMF (PEMF) source, etc.). In certain examples, the human-imperceptible carrier wave can be a visual source configured to provide infrared (IR), ultraviolet (UV), or other light that is not visible to the patient, or an audio source (e.g., a speaker) configured to provide sound waves outside (e.g., above or below) the range of audible human perception. Light is electromagnetic radiation at a specific range of frequencies. Light in the visible spectrum (e.g., between 400 THz and 790 THz) is human perceptible in real-time. In an example, perceptible can mean in real-time to the patient, and not, for example, some amount of time after direct exposure, such as in the case of UV light and skin damage, certain radiation and cancer, etc. The human-imperceptible carrier wave can be modulated with a biological or natural frequency stimulation signal to provide a modulated stimulation signal that is not perceptible to the patient.

An electromagnetic wave is produced by the electromagnetic source. However, a fundamental wavelength of a carrier wave of the electromagnetic wave can be selected such that a person within 10 meters of the source is in the near field of the electromagnetic source. For example, the patient may be in the near field by being less than six wavelengths distance from the source. As a result, each of the embodiments described herein a magnetic field component of the electromagnetic may be dominant compared with an electric field component at the patient. In some embodiments, the patient is orders of magnitude closer to the source than the wavelength. For example, the wavelength of an electromagnetic carrier wave may be in the order of 3 km compared with the person in the order of 3 meters from the source. Thus, sources producing changing magnetic fields, such as a coil receiving AC current, etc., are considered herein to be a subset of electrometric sources.

In certain examples, the modulation disclosed herein can include, among others, one or more of pulse-width modulation (PWM), amplitude modulation (AM), frequency modulation, or other modulation of the carrier wave. The biological or natural frequency stimulation signal can include one or more of: an alpha wave stimulation signal (one example of a biological frequency stimulation signal); a Schumann resonance stimulation signal (one example of a natural frequency stimulation signal); one or more Solfeggio frequencies; or one or more other biological or natural frequency stimulation signals represented as one or more other frequencies, such as disclosed herein. The biological or natural frequency stimulation signal may, in some examples, include a combination of such signals, for example: two or all of an alpha wave frequency; a Schumann resonance frequency; and a Solfeggio frequency. In certain examples, the biological or natural frequency stimulation signal can include a signal having at least one of: a frequency between 110 Hz and 112 Hz; a frequency between 7 Hz and 14 Hz; or a frequency of 528 Hz. In other examples, the biological or natural frequency stimulation signal can comprise one or more other frequencies, such as described herein.

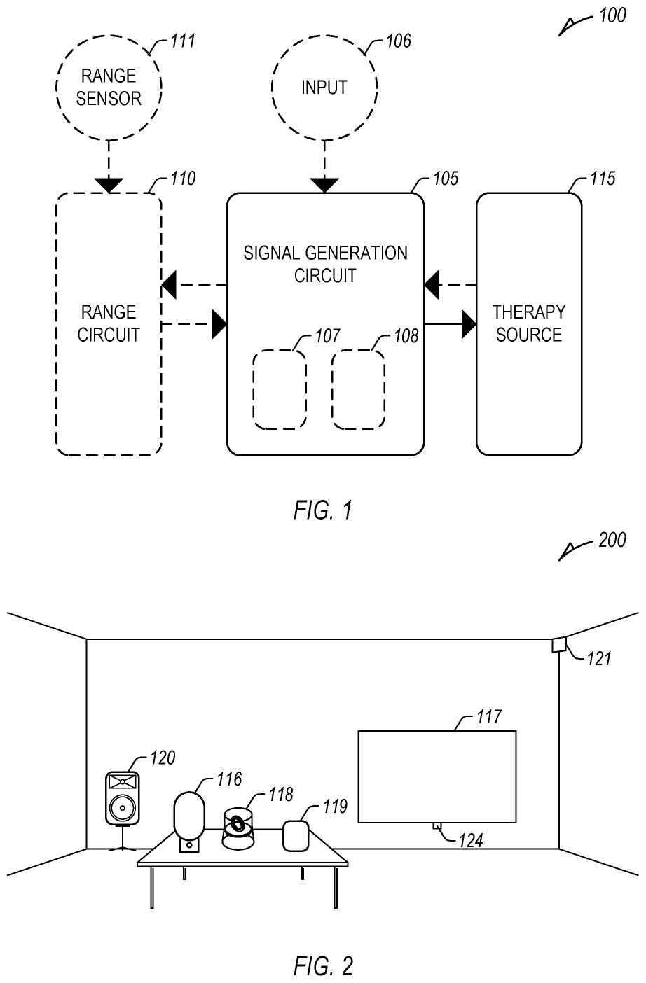

FIG. 1 illustrates an example system 100 for providing a modulated stimulation signal to a patient, including a signal generation circuit 105, a range circuit 110, and a therapy source 115. The signal generation circuit 105 can include a signal generator 107 configured to generate a carrier wave having a carrier frequency, and a modulation circuit 108 configured to modulate the carrier wave. In other examples, the signal generation circuit 105 can receive a carrier wave from one or more other sources (e.g., the therapy source 115, etc.) and modulate the received carrier wave. In certain examples, the signal generation circuit 105 can be configured to receive information, such as from a user through an input 106 (e.g., a user interface, etc.) or from one or more other sources, such as the range circuit 110, the therapy source 115, etc. The signal generation circuit 105 can be configured to modulate the carrier wave with a biological or natural frequency stimulation signal to provide a modulated stimulation signal, such as to be provided to a patient using the therapy source 115. In other examples, the signal generation circuit 105 can be configured to generate or provide the modulated stimulation signal, such as to determine the modulated stimulation signal to generate or provide with respect to a carrier wave and the biological or natural frequency stimulation signal. In certain examples, the biological or natural frequency stimulation signal can include a received or stored alpha-wave signal of the patient or one or more other individuals, and the signal generation circuit 105 can be configured to determine and generate or provide the modulated stimulation signal using the received or stored alpha-wave signal. In other examples, the signal generation circuit 105 can be configured to retrieve the modulated stimulation signal from memory and provide the modulated stimulation signal to the therapy source 115 to be provided to the patient.

The therapy source 115 can include one or more devices configured to apply a signal to the patient, including to provide the modulated stimulation signal to the patient to affect a mood of the patient, such as to induce or increase a pleasant or deactivated state or feeling of calm, relaxed, serene, or content. In an example, the therapy source 115 can include one or more of an audio, visual, or electromagnetic source configured to provide a human-perceptible or human-imperceptible signal, output, or wave. FIG. 2 illustrates different examples of the therapy source 115, including a light 116, a display 117, an electromagnetic therapy source 118, a smart speaker 119, a speaker 120, a motion detector 121, and a companion device 124.

In other examples, the companion device 124 can include a visual source configured to be placed in a common viewing direction of a patient, such as in co-located with the display 117, configured to provide the modulated stimulation signal, such that the patient receives the modulated stimulation signal provided by the companion device while viewing the display 117.

The intensity or magnitude of applied electromagnetic fields can be inversely proportional to the square of the distance from the source. The intensity or magnitude of sound and light also decrease with respect to distance from the source. The present inventors have recognized that the modulated stimulation signal can be adjusted according to the distance between the patient and the therapy source 115.

In an example, the therapy source 115 can include an emitter. The emitter may comprise a coil or a quarter-wave, half-wave, or full-wave antenna. The emitter may be designed, using any known technique, to apply or provide a desired magnitude of an electromagnetic field at the patient's head. The desired magnitude may be in terms of electric field or magnetic field. The magnitude may be designed to be at least as strong as, but in some embodiments stronger than (e.g., at or at least a value that is between 1.5 and 2.5 times stronger)the magnitude that the patient can themselves produce. The magnitude that the patient can themselves produce may be derived experimentally for that person or based on a sample of people by measuring the magnitude of the field at their head. Optionally, the maximum that the person can themselves produce may be taken to be a maximum peak-to-peak value that is observed over a 24, 48, or 72-hour period. In another embodiment the magnitude may be designed to be at least as strong as, but in some embodiments stronger than (e.g., at or at least a value that is between 1.5 and 2.5 times stronger) the magnitude that the patient can themselves produce when they produce alpha waves. It will be understood that the patient's head may be at a range of distances from the emitter. As non-limiting examples, the therapy source 115 can be configured to apply the electromagnetic field to the patient at a distance in the range of 0 to 4 meters from the emitter, or in the range 1 to 3 meters, etc. In a non-limiting example, the therapy source 115 can be configured to apply the electromagnetic field to the patient at a distance that is substantially 2 meters from the therapy source 115 (e.g., the electromagnetic therapy source 118, etc.).

The range circuit 110 can be configured to receive information from a range sensor 111. In certain examples, the range sensor 111 can include one or more of a sonar sensor, a radar sensor, a lidar sensor, an image sensor, a sound sensor, or one or more other sensors configured to receive information indicative of movement or range of a patient or one or more other subjects in a field of view of the range sensor 111. The range sensor 111 can be configured to provide one or more stimulus signals across a field of view and receive a reflection or response to the provided one or more stimulus signals and provide an indication of movement in the field of view. In other examples, the range sensor can only receive information from the range sensor 111. The range circuit 110 can be configured to receive information from the range sensor 111 and determine an indication of range between the therapy source 115 and the object moving in the field of view. In certain examples, the range circuit 110 can be configured to determine one or more characteristic of the object identified as moving in the field of view. In certain examples, the range circuit 110 can be configured to provide the determined range or characteristic to the signal generation circuit 105. The signal generation circuit 105 can be configured to adjust an amplitude or power of the modulated stimulation signal using the determined range or characteristic. For example, when the patient is near to the therapy source, the power or amplitude of the stimulation signal can be decreased, such as to not overstimulate the patient, or to not provide stimulation beyond a determined maximum intensity, etc. When the patient is farther away, the power of the stimulation signal can be increased (e.g., to still provide a therapeutic benefit, etc.).

In other examples, the range sensor 111, or a network of multiple range sensors, can be used to determine of distinguish between one or more other patient characteristic (e.g., size, sex, age, physical body position, voice command, planar relationship to the therapy source, etc.), and the modulated stimulation signal can further be adjusted (e.g., increased or decreased, etc.) using the determined patient characteristic. In certain examples, one or more initial patient characteristics can be input by a user. In an example, the range sensor 111 can be used to distinguish between or among multiple users, or to confirm one or more characteristics of a specific user or patient. In an example, the modulated stimulation signal can be applied if the patient moves to a specific area (e.g., sits in a specific chair, moves to a specific portion of a room, etc.), or takes a specific physical body position (e.g., lies down in bed, etc.). Further, the patient desiring the stimulation signal can have one or more learned or determined characteristics, such as a height or range of heights, a speed of movement, etc., such that the therapy source will not provide therapy until one or more of those learned or determined characteristics are met. In certain examples, the therapy source can be controlled using one or more voice commands, or one or more patient characteristics can be determined by vocal analysis. The therapy source can have different settings depending on the determined patient characteristics.

In an example, the range sensor 111 can have a specific “field of view” or detection range with respect to the space in which the range sensor 111 is placed, such as a room in which the therapy source 115 is placed, etc. The range sensor 111 can have a known position with respect to the therapy source 115. In certain examples, the range sensor 111 can include the therapy source 115. In other examples, the range sensor 111 can include one or multiple devices including or separate from the therapy source 115.

In certain examples, the range circuit 110 can be configured to determine a relative distance between the range sensor 111 and the patient using a determined area of the patient with respect to the field of view of the range sensor 111. For example, the field of view of an image sensor can include a number of pixels, and the relative distance between the image sensor and the patient can be determined with respect to a number of pixels determined as the area of the patient. The area of the patient can be determined with respect to a difference between a current view and a background image, a detected relative change, etc. In certain examples, the relative measures can be determined using a weighted center of received reflections (e.g., analogous to a patient center of mass) or one or more other relative determinations of a position of the patient's head, etc. The weighed center may, for example, be determine by weighting the positions of each reflection by a respective indication of the strength of the reflection. One or more calibration measures can be determined or looked-up, for example, using a look-up table stored in memory, using such relative measures.

Similar methods can be used for other sensors, such as sonar, radar, or lidar sensors. For example, the relative distance between the range sensor 111 and the patient can be determined by a time of return of a portion of provided range signal determined as the area of the patient, such as a detected reflection of an emitted sound, light, or radio waves. Like with the example image sensors, above, the area of the patient can be determined with respect to a difference between a current view and a background image, a detected relative change, etc. The relative area can be determined, such as with maximum and minimum area determined as the patient (e.g., moving area) moves in and out of the field of vision. In other examples, one or more initial thresholds can be determined or assigned. In certain examples, during initialization of the therapy source 115, a user can be instructed to calibrate the range sensor 111, such as by following prompts to stand in known or input locations with respect to the range sensor 111. In an example, one or more companion objects (e.g., stickers, reflectors, known objects, objects of known shapes, etc.) can be placed in the room to calibrate the range sensor 111. For example, the range sensor 111 can include first and second devices having different views of an area. The first and second devices can be of known size or characteristic, such that the devices, once placed, can provide a calibration measure. The range circuit 110 can determine such calibration measures.

In certain examples, the range circuit 110 can be configured to determine a relative angular displacement of the patient with respect to a position, orientation, or axis of the therapy source 115. In addition to relative distance between the range sensor 111 and the patient or the therapy source 115 and the patient, if the therapy source 115 has a non-uniform distribution of applied signal, the range circuit 110 can determine an additional angular calibration measure with respect to the non-uniform distribution. For example, a dipole antenna has a known non-uniform distribution in multiple dimensions. If the therapy source 115 is a dipole antenna, the range circuit 110 can be configured to determine an angular calibration measure with respect to the position of the patient and the position, orientation, or axis of the dipole antenna. In certain examples, the range circuit 110 can be configured to determine one or more measures of the patient, such as relative position in two or three dimensions with respect to the therapy source 115, and determine the calibration measures. In certain examples, the range circuit 110 can compute the calibration measures with respect to known relationships. In other examples, calibration measures can be estimated or retrieved using relative positions and entries from one or more look-up tables stored in memory.

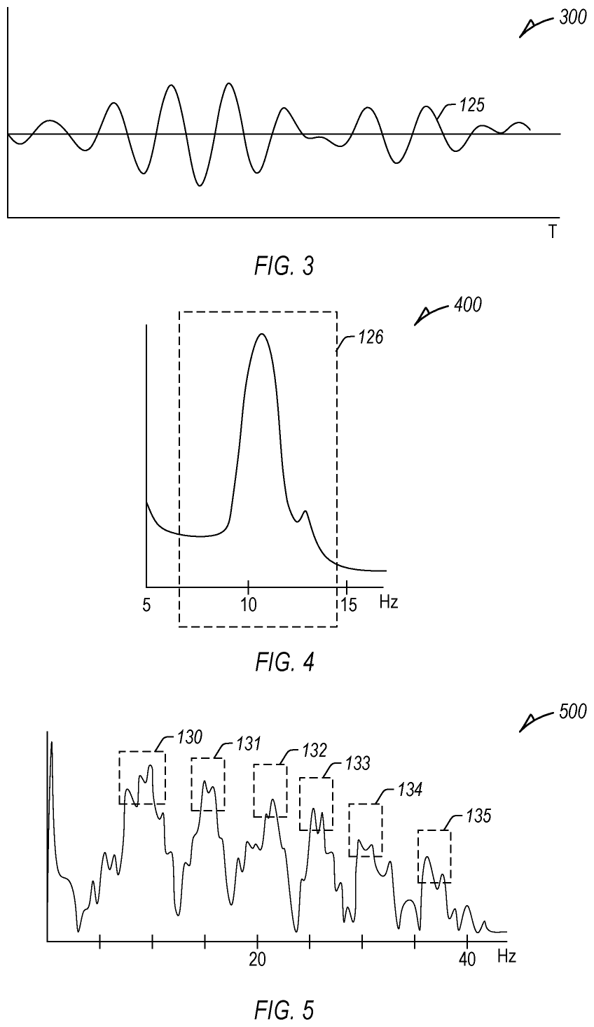

FIGS. 3-4 illustrate example time and frequency domain representations of an electroencephalogram (EEG) signal 300, 400, including an example alpha wave signal 125 in the time domain (T) and a frequency range of the alpha wave 126 in the frequency domain (Hz). Alpha waves are neural oscillations ranging in frequency and amplitude between 7 and 14 Hz (e.g., 8 to 12 Hz, 8 to 13 Hz, etc.) typically associated with a pleasant or deactivated state or feeling of calm, relaxed, serene, or content. However, stimulation signals having the frequency of alpha waves (e.g., an alpha-wave frequency range) can, in certain examples, be perceptible to the patient.

The present inventors have recognized, among other things, that a human-imperceptible stimulation signal can be modulated with an alpha-wave frequency range or other stimulation signal to provide a modulated stimulation signal that, when applied with the therapy source, can induce alpha waves in the patient, providing the benefit of alpha-wave stimulation without the patient perceiving the modulated stimulation signal. In certain examples, the alpha waves described herein can include an alpha wave recorded from the patient or one or more other patients. A digital representation of the alpha wave can be stored, such as in memory, and used, for example, as the biological or natural frequency stimulation signal, to provide the modulated stimulation signal.

Although discussed herein with respect to alpha waves, in other examples, the biological or natural frequency stimulation can alternatively include other neural oscillations, such as delta waves (e.g., 0.5 to 4 Hz), theta waves (e.g., 4 to 8 Hz), beta waves (e.g., 13 to 40 Hz), gamma waves (e.g., 40 Hz to 100 Hz), etc., to provide one or more other mental states associated with such waves. For example, delta waves are associated with deep sleep. In certain examples, delta wave activity can be induced, such as to induce or increase a feeling of sleepiness in the patient or to improve sleep quality. Theta waves are associated with various aspects of cognition and behavior, including learning, memory, and spatial navigation. Beta waves are associated with active consciousness, including busy or anxious thinking and concentration. Gamma waves are associated with processing complex tasks, important for learning, memory, and processing, and are used as a binding tool for senses to process new information. Activity associated with each can be induced, such as to induce or increase feelings or activities associated therewith.

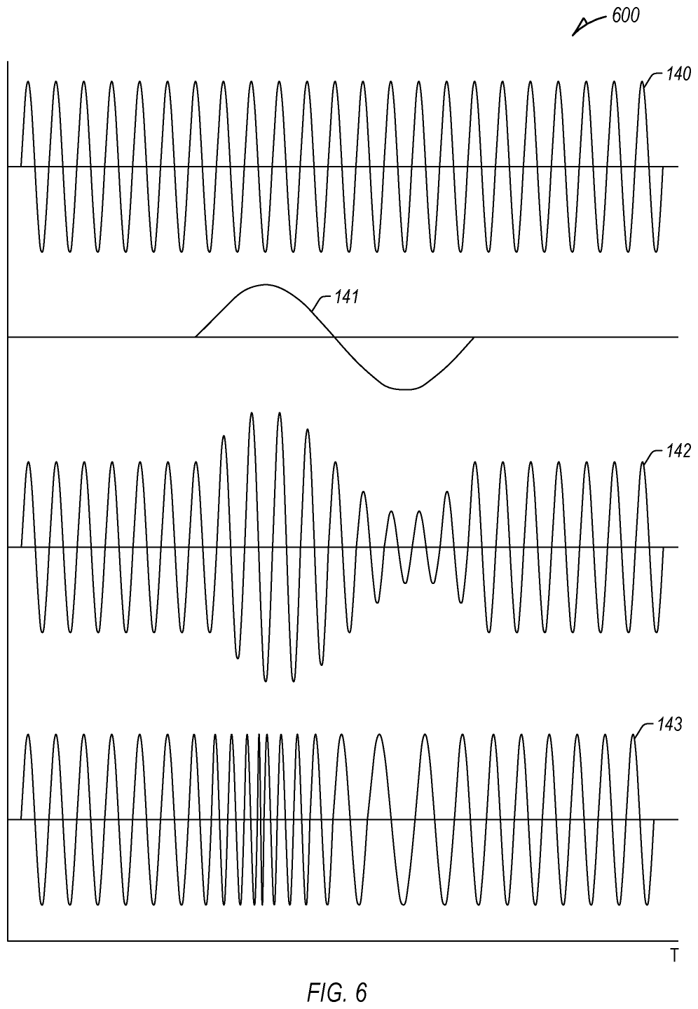

FIG. 5 illustrates an example natural frequency signal 500 including one or more peaks associated with the earth magnetic field (e.g., Schumann resonance, etc.), including a first resonance 130 at about 7.83 Hz, a second resonance 131 at about 14 Hz, a third resonance 132 at about 21 Hz, a fourth resonance 133 at about 26 Hz, a fifth resonance 134 at about 33 Hz, and a sixth resonance 135 at about 39 Hz. In other examples, the example natural frequency stimulation can include multiples thereof, such as 110 Hz to 112 Hz, etc. Other biological or natural stimulation frequencies can include one or more Solfeggio frequencies, such as 396 Hz, 528 Hz, 639 Hz, 741 Hz, or 852 Hz. In an example, combinations of the alpha wave stimulation (or other neural oscillations) and one or more other biological or natural frequency stimulation can be used to modulate the human-imperceptible stimulation signal.

For example, the amplitude of a high-frequency carrier wave (e.g., above 100 kHz, between 100 kHz and 550 kHz, etc.) can be modulated to carry a biological or natural stimulation frequency. In an example, the amplitude of the carrier wave can be modulated to carry a first modulation signal, such as a multiple or octave of the Shuman resonance, e.g., 110 to 112 Hz. In other examples, the amplitude of the carrier wave can be modulated, such as sweeping between first and second boundaries of a frequency range of the first modulation signal, e.g., 110 to 112 Hz, by a second modulation signal, such as the Shuman resonance (e.g., 7.86 to 8 Hz), alpha waves (e.g., 7 to 14 Hz), etc. In other examples, one or more other stimulation signals or combinations of stimulations signals can be used, combined, swept, etc.

FIG. 6 illustrates example signal modulation 600 including modulating a carrier wave 140 by a stimulation signal 141 to produce a first and second example modulated stimulation signals 142, 143. In this example, the carrier wave 140 has a constant carrier frequency and the stimulation signal 141 includes a simple sine wave having a constant, lower frequency than the carrier wave 140.

In an example, the modulation can include amplitude modulation. The amplitude of the carrier wave 140 can be modulated by the stimulation signal 141 to produce the first example modulated stimulation signal 142.

In other examples, modulation can include one or more other types of signal modulation, such as frequency modulation, pulse width modulation (PWM), etc. For example, the frequency of the carrier wave 140 can be modulated by the stimulation signal 141 to produce the second example modulated stimulation signal 143. In other examples, one or more stimulations types can be combined, such as amplitude modulation and PWM, frequency modulation and PWM, etc.

In other examples, one or more of the carrier wave 140 or the stimulation signal 141 can include signals having changing amplitude or frequency. In an example, the carrier wave 140 can be modulated across a first range of frequencies of a first stimulation signal, such as over an alpha-wave frequency range (e.g., between 7 Hz and 14 Hz), a range of or about a multiple of the Schumann resonance (e.g., 110 Hz to 112 Hz), etc. The rate at which the modulation varies across the first range of frequencies can be controlled by a second stimulation signal, such as a frequency of the second stimulation signal, or across a second frequency range of the second stimulation signal. For example, the first stimulation signal can include a natural stimulation signal, such as a multiple of the Schumann resonance (e.g., 110 Hz to 112 Hz), and the second stimulation signal can include a biological stimulation signal, such as an alpha-wave signal (e.g., 10 Hz, 8 Hz to 13 Hz, 7 Hz to 14 Hz, etc.).

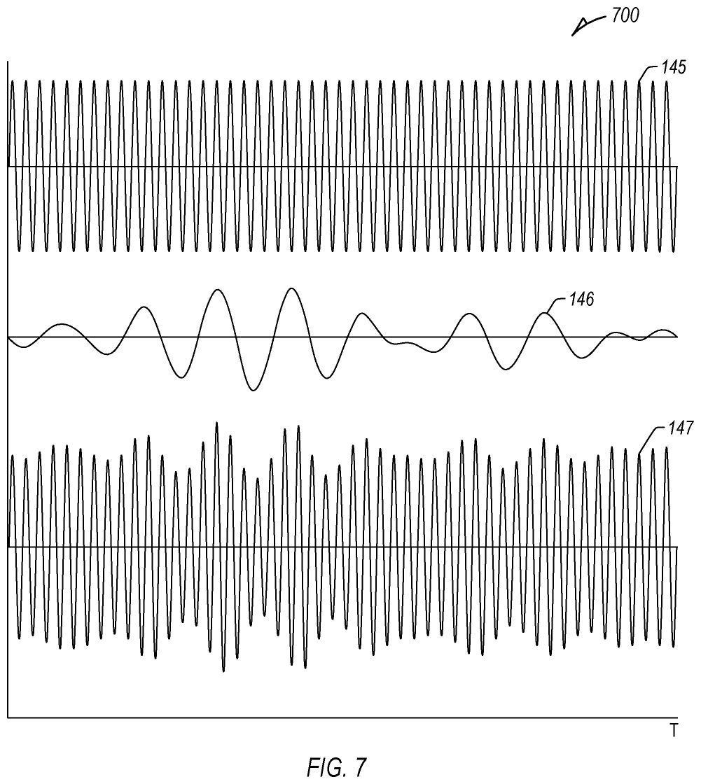

FIG. 7 illustrates example signal modulation 700 including modulating a carrier wave 145 by a stimulation signal 146 to produce a third example modulated stimulation signal 147. In this example, the carrier wave 145 has a constant carrier frequency and the stimulation signal 146 includes a biological or natural frequency signal, in this example a complex signal representative of an alpha wave, varying in frequency and amplitude. In other examples, the stimulation signal 146 can include one or more other biological or natural frequency signals.

In the example of FIG. 7, the amplitude of the carrier wave 145 is modulated by the stimulation signal 146. In other examples, one or more other forms of modulation can be performed. In an example, the carrier wave 145 can include one or more other signals of constant or varying amplitude, frequency, or other characteristic (e.g., square wave instead of sine wave, triangle wave, sawtooth wave, PWM signal, etc.).

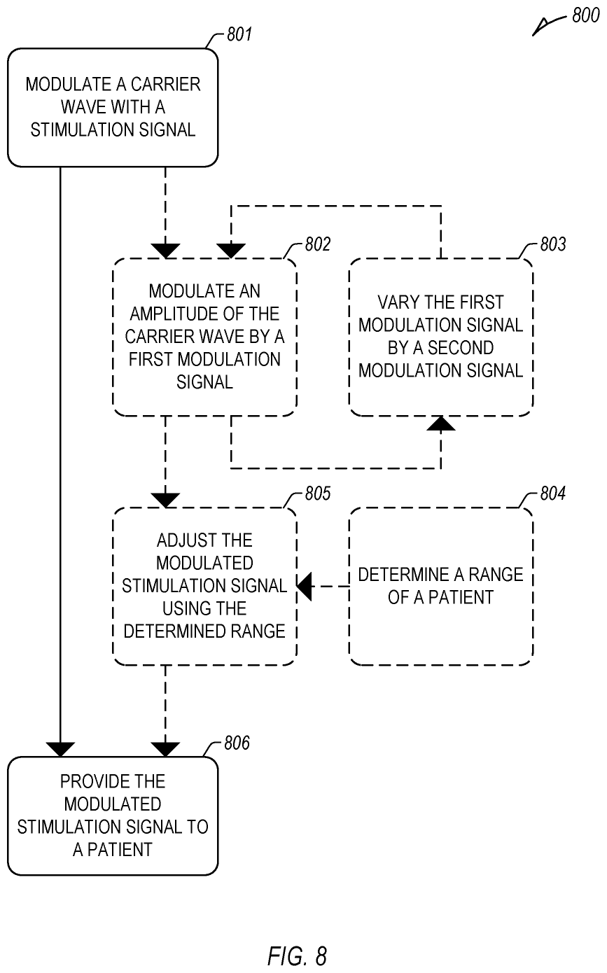

FIG. 8 illustrates an example method 800 of inducing alpha-wave activity in a patient, or providing alpha-wave stimulation to the patient. At 801, a carrier wave can be modulated with a biological or natural frequency stimulation signal to provide a modulated stimulation signal, such as using a signal generation circuit. In an example, the carrier wave can have a fixed or varying frequency and a fixed or varying amplitude.

At 802, an amplitude of the carrier wave can be modulated by a first modulation signal. The first modulation signal can include a signal having a fixed or varying frequency or range of frequencies. In other examples, the modulation can include pulse-width modulation (PWM), frequency modulation, or combinations of two or more types of modulation.

At 803, the first modulation signal can be varied by a second modulation signal. In an example, the second modulation signal can have a fixed or varying frequency or range of frequencies. In an example, one or both of the first and second stimulation signals can be a biological or natural frequency stimulation signal, such as disclosed herein. In an example, modulating the first stimulation signal about a single frequency, or across a range of frequencies, can improve the likelihood that an effective stimulation signal is applied to the patient. When both the first and second stimulation signals are biological or natural frequency stimulation signals, the likelihood that the patient is positively affected is improved. In other examples, the first modulation signal can be varied randomly about a frequency or range of frequencies.

At 804, a range of a patient from a therapy source can be determines, such as suing a range sensor and range circuit, such as described above. At 805, the modulated stimulation signal can be adjusted using the determined range. At 806, the modulated stimulation signal, and in certain examples, the adjusted modulated stimulation signal, can be provided to the patient, such as using the therapy source.

In addition to or instead of amplitude modulation (AM), the modulation described herein can include amplitude key shifting (ASK) modulation, for example, on-off keying. For example, on-off keying can include 0% or 100% modulation of the carrier wave using a square wave. Such implementations are therefore also within the scope of the presentation, for example as a substitute for amplitude modulation.

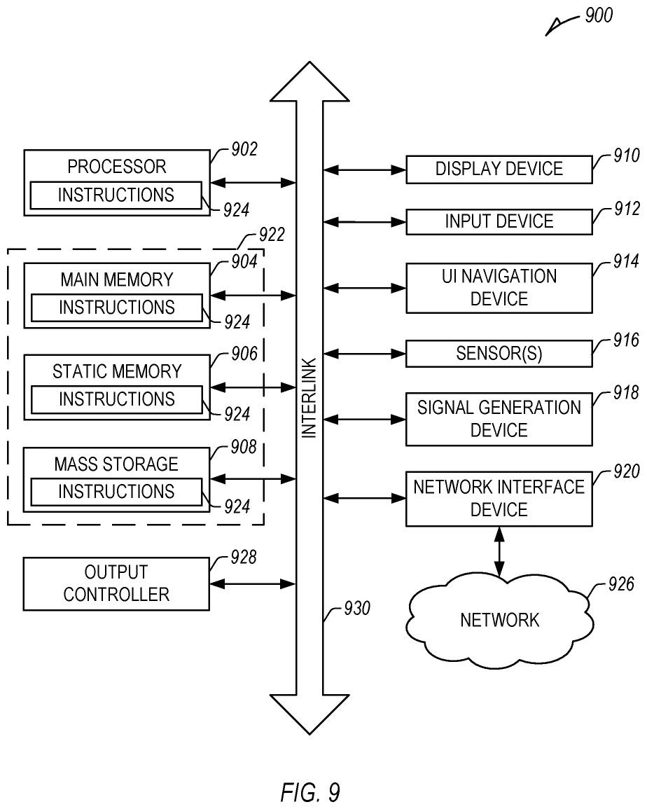

FIG. 9 illustrates a block diagram of an example machine 900 upon which any one or more of the techniques (e.g., methodologies) discussed herein may perform. Portions of this description may apply to the computing framework of one or more of the dosimeters, circuits, or processors described herein. Examples, as described herein, may include, or may operate by, logic or a number of components, or mechanisms in the machine 900. Circuitry (e.g., processing circuitry, a dosimeter circuit, etc.) is a collection of circuits implemented in tangible entities of the machine 900 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 900 follow.

In alternative embodiments, the machine 900 may operate as a standalone device or may be connected (e.g., networked) to other machines. In a networked deployment, the machine 900 may operate in the capacity of a server machine, a client machine, or both in server-client network environments. In an example, the machine 900 may act as a peer machine in peer-to-peer (P2P) (or other distributed) network environment. The machine 900 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 900 may include a hardware processor 902 (e.g., a central processing unit (CPU), a graphics processing unit (GPU), a hardware processor core, or any combination thereof), a main memory 904, a static memory (e.g., memory or storage for firmware, microcode, a basic-input-output (BIOS), unified extensible firmware interface (UEFI), etc.) 906, and mass storage 908 (e.g., hard drive, tape drive, flash storage, or other block devices) some or all of which may communicate with each other via an interlink (e.g., bus) 930. The machine 900 may further include a display unit 910, an alphanumeric input device 912 (e.g., a keyboard), and a user interface (UI) navigation device 914 (e.g., a mouse). In an example, the display unit 910, input device 912, and UI navigation device 914 may be a touch screen display. The machine 900 may additionally include a signal generation device 918 (e.g., a speaker), a network interface device 920, and one or more sensors 916, such as a global positioning system (GPS) sensor, compass, accelerometer, or one or more other sensors. The machine 900 may include an output controller 928, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 902, the main memory 904, the static memory 906, or the mass storage 908 may be, or include, a machine-readable medium 922 on which is stored one or more sets of data structures or instructions 924 (e.g., software) embodying or utilized by any one or more of the techniques or functions described herein. The instructions 924 may also reside, completely or at least partially, within any of registers of the processor 902, the main memory 904, the static memory 906, or the mass storage 908 during execution thereof by the machine 900. In an example, one or any combination of the hardware processor 902, the main memory 904, the static memory 906, or the mass storage 908 may constitute the machine-readable medium 922. While the machine-readable medium 922 is illustrated as a single medium, the term “machine-readable medium” may include a single medium or multiple media (e.g., a centralized or distributed database, and/or associated caches and servers) configured to store the one or more instructions 924.

The term “machine-readable medium” may include any medium that is capable of storing, encoding, or carrying instructions for execution by the machine 900 and that cause the machine 900 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 924 may be further transmitted or received over a communications network 926 using a transmission medium via the network interface device 920 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 920 may include one or more physical jacks (e.g., Ethernet, coaxial, or phone jacks) or one or more antennas to connect to the communications network 926. In an example, the network interface device 920 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 900, and includes digital or analog communications signals or other intangible medium to facilitate communication of such software. A transmission medium is a machine-readable medium.

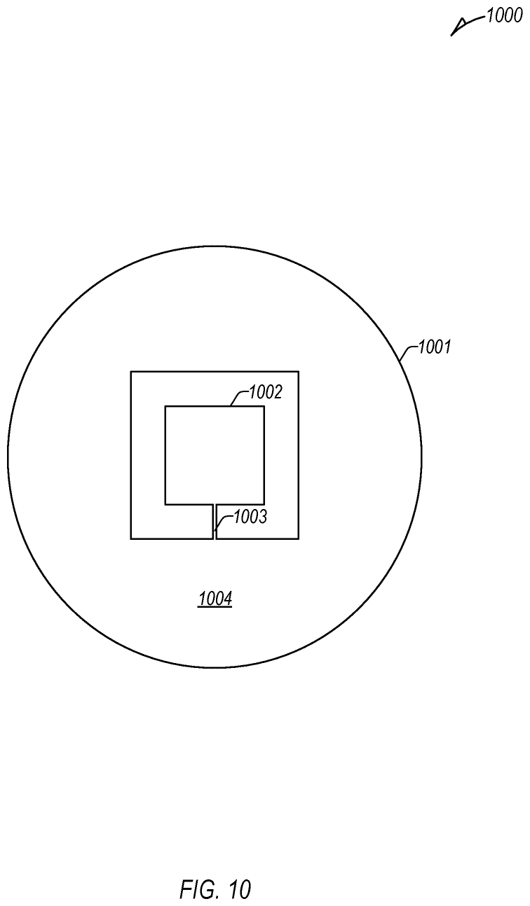

FIG. 10 illustrates an example layout 1000 of a front surface of a PCB 1001 for a therapy device including an antenna 1002. In an example, the antenna 1002 can include a directional antenna, such as a patch antenna, in certain examples printed or otherwise positioned on the PCB 1001, such as centrally located (e.g., printed) on the front surface of the PCB 1001. The antenna 1002 can include a feed 1003 across a gap to a larger ground plane 1004. To maintain a wide field of view, a single patch antenna may be used, such as opposed to an array of patch antennas or a stacked patch antenna, etc.

In certain examples, the PCB 1001 can include one or more ground planes for the antenna 1002, such as located on one or both sides of the PCB 1001. Although discussed herein with respect to a patch antenna, in other examples, the antenna 1002 can include one or more other directional antennas, such as an array of multiple antenna elements or one or more antenna elements having a waveguide or reflector, etc.

Although illustrated in FIG. 10 as having only the antenna 1002, the feed 1003, and the ground plane 1004, in other examples, the PCB 1001 can include one or more other circuits or components coupled to or placed on the front surface of the PCB 1001. However, in certain examples, the amount of conductive material or additional components on the front surface can be limited so as to minimize interferences with the antenna 1002. In an example, one or more other circuits or components, such as power electronic components, driver circuits, or one or more components having dense conductive material (e.g., shielded components, heat sinks, etc.) can be required to be placed on the back surface of the PCB 1001, such as to reduce interference with electromagnetic radiation emitted from the antenna 1002. Additionally, such components can be located away from the center of the back surface of the PCB 1001 (e.g., opposite the antenna 1002 and in certain examples the isolation region between the antenna 1002 and the ground plane illustrated on the front surface) and in certain examples additionally balanced (e.g., by density of conductive material, by current density, etc.) about the back surface to further reduce interference with antenna performance. In an embodiment, a tallest component on the back of the PCB 1001 is taller than a tallest component on the front of the PCB 1001. For example, in an embodiment, no component on the back of the PCB 1001 has a height greater than 10mm. The components on the back of the PCB 1001 may include one or more or all of a voltage-controlled oscillator for generating a non-visible electromagnetic wave carrier frequency as described herein, a supercapacitor, and/or an inductor.

In certain examples, the patch antenna can provide a number of advantages, including , among others, directional transmission to provide a controllable or desired field of view and, in certain examples, such as in combination with a ground plane, a controllable or desired front-to-back ratio. In certain examples, the patch antenna can be printed or otherwise provided on a surface of the board (e.g., on-board), at a reduced cost in contrast with other antenna structures or separate antenna circuits or components, with a reduced size, thickness, or width of the board supporting the on-board patch antenna, providing a final assembled device with a smaller device profile, for example, in contrast to separate antenna circuits, etc.

In an example, the frequency of the electromagnetic field therapy (e.g., a carrier frequency, etc.) can include a frequency in one or more Wi-Fi ranges (e.g., 2.4 GHz, 5 GHz, 5.7-5.8 GHz, etc.), or close thereto (e.g., within a threshold percentage, such as 10, etc.). In certain examples, being close to a Wi-Fi range has the advantage of providing a carrier wave at a frequency common or recognizable to the user.

The field of view for an antenna (without consideration of other electronic components proximate to the antenna, such as on a PCB of a finished therapy device, etc.) can include a field of view with respect to a major lobe of a main beam of radio frequency radiation having exposure to radiation above a threshold therapy tolerance (e.g., −3 dB beamwidth (BW), i.e., the field of view of the antenna can be defined as the half-power beamwidth (HPBW) of the radiation pattern).

One or more control circuits can limit the radiation a user is exposed to using the HPBW and the efficiency (or peak power) of the antenna, such as by controlling the transmit power of a transmitter coupled to the antenna, etc. In certain examples, the power provided to users can be within or below a safety limit published or set by one or more standards bodies or government agencies responsible for setting or enforcing standards for communication devices in one or more jurisdictions, such as the Federal Communications Commission (FCC) (e.g., FCC limit for public exposure of 1.6 watts per kilogram (W/kg) or one or more other limits, such as 10 uW/m2, 0.05 V/m, etc.), the European Telecommunications Standards Institute (ETSI) (e.g., ETSI limits of 20, 23, 30, or 36 dBm, or 100, 200, 1000, or 4000 mW, etc.), etc. In certain examples, there may be no limitations on transmitted power, depending on a frequency of the carrier wave and its harmonic frequencies. In other examples, one or more limitations can be imposed on one or more harmonic frequencies of the carrier wave, while no limitations or fewer limitations are placed on the carrier wave itself.

In certain examples, the shape of the major lobe of the main beam can be impacted by other components of the therapy device, such as power electronic components (e.g., HV capacitors, etc.), one or more processors or processing circuits or components thereof (e.g., heat sinks, regulators, etc.), etc. As used herein, the major lobe can refer to the radiation pattern of the antenna alone, without consideration of other components of the therapy device. In other examples, the major lobe can separately refer to the radiation pattern of the antenna in the finished therapy device, after considering interference from all other components.

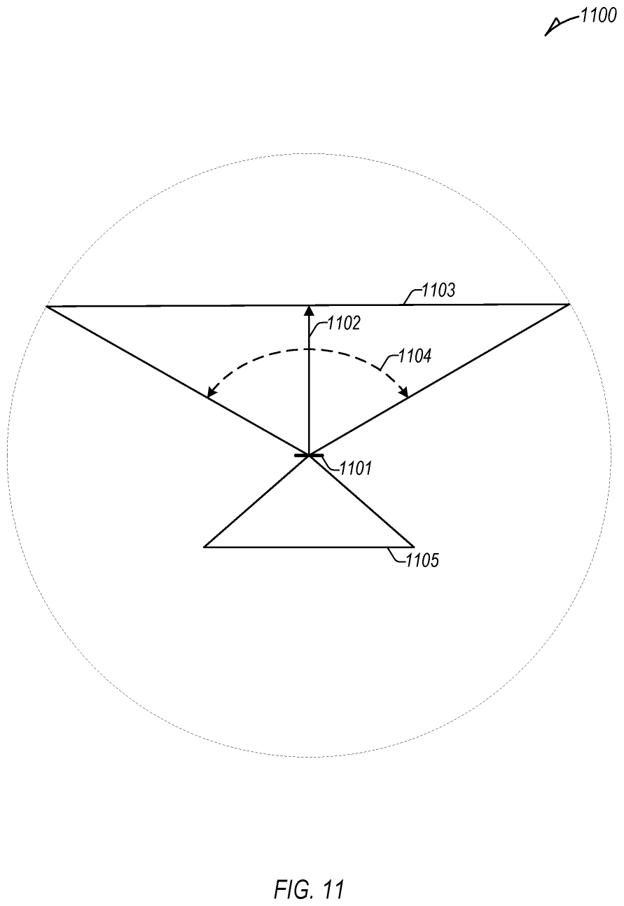

FIG. 11 illustrates example measurements 1100 for a therapy device 1101 (e.g., illustrative and not necessarily according to scale) with respect to a major stimulation axis 1102 of a patch antenna, the measurements including an antenna field of view 1103 having a first angle 1104 (e.g., between 90 and 155 degrees, between 120 and 155 degrees, 120 degrees, 130 degrees, 150 degrees, etc.). The example measurements 1100 illustrated in FIG. 11 are with respect to the antenna without consideration of interference from other electronics of a finished therapy device (e.g., power electronics, driver circuits, amplifiers, regulators, processors or processing circuits, memory devices, etc.). Accordingly, a finished therapy device 1101 including all components can produce field variation differences, such as by interference with the theoretical or tested electromagnetic radiation introduced by other conductors or electric or magnetic fields in the housing of the therapy device 1101 (e.g., heat sinks for electronic components, etc.).

In certain examples, the range (e.g., distance) of the antenna field of view 1103 can be dependent upon, among other things, one or more of a transmitter or signal generator power provided to the antenna, electrical characteristics of a matching circuit, the presence of a ground plane or other shielding elements, etc.

In addition to the antenna field of view 1103, the therapy device 1101 can additionally have a back field of view 1106. In an example, the front-to-back ratio of the strength of the electromagnetic field radiated along the major stimulation axis 1102 compared to that radiated in the back direction (e.g., a front-to-back power ratio) can be greater than 10-to-1, or in certain examples even greater (e.g., 12-, 13-, 14-to-1, etc.).

The example measurements 1100 described herein are based on testing of a centrally-located printed 5.8 GHz patch antenna on a PCB (such as illustrated in FIG. 10) in the therapy device 1101 (such as illustrated in FIGS. 14-17), providing an antenna efficiency of 30.9% (±10%) (e.g., 30.9 mW, 14.9 dBm, −5.1 dB, 1 dBi, such as from a 100 mW signal generator), a horizontal (azimuth, as viewed from above) antenna field of view (e.g., −3 dB BW, HPBW, etc.) of 120 degrees relative to the major stimulation axis 1102, a vertical (elevation, as viewed from the side) antenna field of view (e.g., −3 dB BW, HPBW, etc.) of 150 degrees relative to the major stimulation axis 1102, a voltage standing wave ratio (VSWR) of 3.0:1 (controlled by the antenna and the matching circuit, which can be selectively detuned by a control circuit or one or more other components, such as to adjust a range of the electromagnetic field therapy, separate from or in addition to adjusting a power of the transmitter or signal generator, etc.), a front-to-back ratio of at least 10 dB, and an input impedance of 50 ohm.

While the example measurements 1100 are illustrative, in certain examples, one or more parameters or measurements described or illustrated herein can depend on various factors, such as environmental factors, etc. (e.g., the surface on which the therapy device 1101 is located at the time of therapy, etc.). For example, positioning the therapy device 1101 on a metal ground plane (e.g., a large metal surface, etc.) can provide a reduced horizontal (azimuth, as viewed from above) antenna field of view (e.g., −3 dB BW, HPBW, etc.) of 100 degrees relative to the major stimulation axis 1102 and a reduced vertical (elevation, as viewed from the side) antenna field of view (e.g., −3 dB BW, HPBW, etc.) of 120 degrees relative to the major stimulation axis 1102. In certain examples, the threshold percentage, alignment, or other parameters or measurements described herein are described with respect to the therapy device resting on a wood or plastic surface and operating in substantially free space, without impeding environmental factors.

Further, as illustrated in the measurements above, the vertical (elevation, as viewed from the side) antenna field of view can be greater than the horizontal (azimuth, as viewed from above) antenna field of view. In other examples, the vertical antenna field of view can be the same as or substantially similar (e.g., within a threshold percentage, such as 5%, etc.) to the horizontal antenna field of view.

In addition, although described and illustrated herein as a therapy device having a base to support the housing on a horizontal surface, in other examples, a wall or ceiling mounted device, or a standing floor lamp device can be anticipated, with the field of view and major stimulation axis relative to an expected location of the user configured to receive the provided stimulation, consistent with the teachings illustrated herein.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the examples herein. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

Included below are further examples, each of which may independently define an aspect or embodiment of the invention.

Example 1 is a system comprising: a signal generation circuit configured to provide a modulated stimulation signal, the modulated stimulation signal including a carrier wave modulated with a biological or natural frequency stimulation signal across a first frequency range of a first modulation signal according to a second modulation signal; and a therapy source configured to provide the modulated stimulation signal to a patient, the modulated stimulation signal configured to provide the benefit of the biological or natural frequency stimulation signal to the patient without the patient perceiving the biological or natural frequency stimulation signal.

In Example 2, the subject matter of Example 1 includes, wherein the system is configured to induce alpha wave activity in the patient, and wherein the therapy source is configured to provide the modulated stimulation signal to the patient to affect a mood of the patient.

In Example 3, the subject matter of Examples 1-2 includes, wherein the system is configured to induce neurological activity in the patient, the neurological activity comprising one or more of alpha wave or delta wave activity in the patient, and wherein the therapy source is configured to provide the modulated stimulation signal to the patient to induce sleepiness or a relaxed state of the patient.

In Example 4, the subject matter of Examples 1-3 includes, wherein the therapy source comprises one of an audio, visual, electric, or electromagnetic source configured to provide the modulated stimulation signal to the patient.

In Example 5, the subject matter of Example 4 includes, wherein the therapy source comprises the audio source configured to provide the modulated stimulation signal to the patient to affect a mood of the patient, and wherein the biological or natural frequency stimulation signal comprises a signal having a frequency between 20 Hz and 20 kHz, corresponding to a perceptible human range of hearing.

In Example 6, the subject matter of Examples 4-5 includes, wherein the therapy source comprises the visual source configured to provide the modulated stimulation signal to the patient to affect a mood of the patient, and wherein the biological or natural frequency stimulation signal comprises a signal having a frequency below 400 Hz corresponding to a range of frequencies where visual changes are human perceptible.

In Example 7, the subject matter of Example 6 includes, wherein the biological or natural frequency stimulation signal comprises a signal having a frequency below 90 Hz corresponding to a range of frequencies where visual changes are human perceptible.

In Example 8, the subject matter of Example 7 includes, wherein the biological or natural frequency stimulation signal comprises a signal having a frequency below 60 Hz corresponding to a range of frequencies where visual changes are human perceptible.

In Example 9, the subject matter of Examples 4-8 includes, wherein the therapy source comprises the electromagnetic source configured to provide the modulated stimulation signal to the patient to affect a mood of the patient.

In Example 10, the subject matter of Example 9 includes, wherein the electromagnetic source comprises a coil.

In Example 11, the subject matter of Examples 9-10 includes, wherein the electromagnetic source comprises a quarter-wave, half-wave, or full-wave antenna.

In Example 12, the subject matter of Examples 1-11 includes, wherein the carrier wave is imperceptible to the patient, and wherein the biological or natural frequency stimulation signal, once modulated onto the carrier wave, is not perceptible to the patient, wherein not perceptible comprises outside a range of human perception.

In Example 13, the subject matter of Examples 1-12 includes, wherein the carrier wave has a frequency corresponding to a range of human perception, and wherein the biological or natural frequency stimulation signal, once modulated onto the carrier wave, is not perceptible to the patient.

In Example 14, the subject matter of Examples 1-13 includes, wherein the modulated stimulation signal comprises the carrier wave amplitude modulated across the first frequency range at a frequency of the second modulation signal.

In Example 15, the subject matter of Examples 1-14 includes, wherein the second modulation signal has a second frequency range, and wherein the modulated stimulation signal comprises the carrier wave amplitude modulated across the first frequency range by the second frequency range.

In Example 16, the subject matter of Example 15 includes, wherein the first and second frequency ranges of the first and second modulation signals, when provided by the therapy source, are within ranges of human perception, and wherein the first and second frequency ranges of the first and second modulation signals, once modulated onto the carrier wave, are not perceptible to the patient.

In Example 17, the subject matter of Examples 15-16 includes, wherein the second frequency range comprises an alpha-wave frequency range.

In Example 18, the subject matter of Examples 15-17 includes, wherein the first frequency range of the first modulation signal is between 110 Hz and 112 Hz, and wherein the second frequency range of the second modulation signal is between 7 Hz and 14 Hz.

In Example 19, the subject matter of Examples 1-18 includes, wherein the carrier wave is within a frequency range comprising an upper bound of 550 kHz.

In Example 20, the subject matter of Examples 1-19 includes, wherein the carrier wave is within a frequency range comprising a lower bound of 100 kHz.

In Example 21, the subject matter of Examples 1-20 includes, wherein the carrier wave comprises a frequency between 100 kHz and 550 kHz.

In Example 22, the subject matter of Examples 1-21 includes, a range sensor; and a range circuit configured to determine a range of the patient from the therapy source using information from the range sensor, wherein the signal generation circuit is configured to adjust the modulated stimulation signal using the determined range.

In Example 23, the subject matter of Examples 1-22 includes, wherein the signal generation circuit is configured to modulate the carrier wave with the biological or natural frequency stimulation signal, including to modulate the carrier wave across the first frequency range of the first modulation signal according to the second modulation signal.

In Example 24, the subject matter of Examples 1-23 includes, wherein the modulated stimulation signal comprises an amplitude modulated signal.

In Example 25, the subject matter of Examples 1-24 includes, wherein the modulated stimulation signal comprises an amplitude key shifting modulated signal.

In Example 26, the subject matter of Examples 1-25 includes, a memory to store the modulated stimulation signal, wherein the signal generation circuit is configured to receive the modulated stimulation signal from memory and to provide the modulated stimulation signal to the therapy source.

In Example 27, the subject matter of Example 26 includes, wherein the biological or natural frequency stimulation signal comprises an alpha-wave signal recorded from the patient or one or more other individuals.

In Example 28, the subject matter of Example 27 includes, wherein the alpha-wave signal comprises an alpha-wave signal of the patient.

Example 29 is a method comprising: providing a modulated stimulation signal using a signal generation circuit, the modulated stimulation signal including a carrier wave modulated with a biological or natural frequency stimulation signal across a first frequency range of a first modulation signal according to a second modulation signal; and providing the modulated stimulation signal to a patient using a therapy source to provide the benefit of the biological or natural frequency stimulation signal to the patient without the patient perceiving the biological or natural frequency stimulation signal.

In Example 30, the subject matter of Example 29 includes, wherein providing the modulated stimulation signal to the patient using a therapy source comprises using one of an audio, visual, or electromagnetic source configured to provide the modulated stimulation signal to the patient.

In Example 31, the subject matter of Examples 29-30 includes, wherein the carrier wave is imperceptible to the patient, and wherein the biological or natural frequency stimulation signal, once modulated onto the carrier wave, is not perceptible to the patient, wherein not perceptible comprises outside a range of human perception.

In Example 32, the subject matter of Examples 29-31 includes, wherein the carrier wave has a frequency corresponding to a range of human perception, and wherein the biological or natural frequency stimulation signal, once modulated onto the carrier wave, is not perceptible to the patient.

In Example 33, the subject matter of Examples 29-32 includes, wherein the modulated stimulation signal comprises the carrier wave amplitude modulated across the first frequency range at a frequency of the second modulation signal.

In Example 34, the subject matter of Examples 29-33 includes, wherein the second modulation signal has a second frequency range, and wherein the modulated stimulation signal comprises the carrier wave amplitude modulated across the first frequency range by the second frequency range.

In Example 35, the subject matter of Example 34 includes, wherein the first and second frequency ranges of the first and second modulation signals, when provided by the therapy source, correspond to ranges of human perception, and wherein the first and second frequency ranges of the first and second modulation signals, once modulated onto the carrier wave, are not perceptible to the patient.

In Example 36, the subject matter of Examples 34-35 includes, wherein the first frequency range of the first modulation signal is between 110 Hz and 112 Hz, and wherein the second frequency range of the second modulation signal is between 7 Hz and 14 Hz.

In Example 37, the subject matter of Examples 29-36 includes, determining a range of the patient from the therapy source using information from a range sensor; and adjusting the modulated stimulation signal using the determined range.

In Example 38, the subject matter of Examples 29-37 includes, modulating the carrier wave with the biological or natural frequency stimulation signal using the signal generation circuit, including modulating the carrier wave across the first frequency range of the first modulation signal according to the second modulation signal.

Example 39 is a system to induce alpha-wave activity in a patient, comprising: a signal generation circuit configured to provide a modulated stimulation signal, the modulated stimulation signal including a carrier wave having a carrier frequency between 100 kHz and 550 kHz modulated with a biological or natural frequency stimulation signal, including across a first frequency range of a first modulation signal according to a second modulation signal; and an electromagnetic therapy source configured to provide the modulated stimulation signal to induce a voltage across cells of the patient to affect a mood of the patient, the modulated stimulation signal configured to provide the benefit of the biological or natural frequency stimulation signal to the patient without the patient perceiving the biological or natural frequency stimulation signal, wherein the first frequency range of the first modulation signal is between 110 Hz and 112 Hz, and wherein the second modulation signal comprises an alpha-wave signal having a frequency between 7 Hz and 14 Hz.

In Example 40, the subject matter of Example 39 includes, wherein the signal generation circuit is configured to modulate an amplitude of the carrier wave with the biological or natural frequency stimulation signal, including to modulate the amplitude of the carrier wave across the first frequency range of the first modulation signal according to the second modulation signal.

In Example 41, the subject matter of Example 40 includes, wherein to modulate the amplitude of the carrier wave across the first frequency range according to the second modulation signal comprises to modulate the amplitude of the carrier wave across the first frequency range at a frequency of the second modulation signal.

In Example 42, the subject matter of Examples 40-41 includes, wherein to modulate the amplitude of the carrier wave across the first frequency range according to the second modulation signal comprises to modulate the amplitude of the carrier wave across the first frequency range by a frequency range of the second modulation signal.

In Example 43, the subject matter of Examples 39-42 includes, a range sensor; and a range circuit configured to determine a range of the patient from the therapy source using information from the range sensor, wherein the signal generation circuit is configured to adjust the modulated stimulation signal using the determined range.

Example 44 is a method to induce alpha-wave activity in a patient, comprising: providing a modulated stimulation signal using a signal generation circuit, the modulated stimulation signal including a carrier wave having a carrier frequency between 100 kHz and 550 kHz modulated with a biological or natural frequency stimulation signal, including across a first frequency range of a first modulation signal according to a second modulation signal; and providing the modulated stimulation signal to induce a voltage across cells of the patient using an electromagnetic therapy source to affect a mood of the patient, the modulated stimulation signal configured to provide the benefit of the biological or natural frequency stimulation signal to the patient without the patient perceiving the biological or natural frequency stimulation signal, wherein the first frequency range of the first modulation signal is between 110 Hz and 112 Hz, and wherein the second modulation signal comprises an alpha-wave signal having a frequency between 7 Hz and 14 Hz.

In Example 45, the subject matter of Example 44 includes, modulating the carrier wave with the biological or natural frequency stimulation signal using the signal generation circuit, including modulating an amplitude of the carrier wave across the first frequency range of the first modulation signal according to the second modulation signal.

In Example 46, the subject matter of Example 45 includes, wherein modulating the amplitude of the carrier wave across the first frequency range according to the second modulation signal comprises modulating the amplitude of the carrier wave across the first frequency range at a frequency of the second modulation signal.

In Example 47, the subject matter of Examples 45-46 includes, wherein modulating the amplitude of the carrier wave across the first frequency range according to the second modulation signal comprises to modulate an amplitude of the carrier wave across the first frequency range by a frequency range of the second modulation signal.

Example 48 is a system to induce alpha-wave activity in a patient, comprising: a signal generation circuit configured to provide a modulated stimulation signal, the modulated stimulation signal including a carrier wave modulated with a biological or natural frequency stimulation signal; and a visual source configured to provide the modulated stimulation signal to the patient to affect a mood of the patient, the modulated stimulation signal configured to provide the benefit of the biological or natural frequency stimulation signal and the carrier wave to the patient without the patient perceiving the biological or natural frequency stimulation signal, wherein the carrier wave and the modulated stimulation signal have frequencies corresponding to a range of human perception, and wherein the biological or natural frequency stimulation signal comprises a stimulation signal having a frequency greater than 100 Hz and less than 500 kHz, corresponding to a range of frequency where visual changes are not human perceptible.

In Example 49, the subject matter of Example 48 includes, wherein the biological or natural frequency stimulation signal has a frequency greater than 400 Hz and less than 500 kHz.

In Example 50, the subject matter of Example 49 includes, wherein the biological or natural frequency stimulation signal has a frequency of 528 Hz.

In Example 51, the subject matter of Examples 48-50 includes, wherein the visual source comprises a light source, and wherein the carrier wave has a carrier frequency between 400 THz and 790 THz, corresponding to a range of human visible light.

In Example 52, the subject matter of Example 51 includes, wherein the light source comprises a room or accent light.

In Example 53, the subject matter of Examples 51-52 includes, wherein the light source comprises a projector or a display, and wherein the carrier wave comprises a portion of an image signal.

In Example 54, the subject matter of Examples 48-53 includes, wherein the signal generation circuit is configured to modulate the carrier wave with the biological or natural frequency stimulation signal.

In Example 55, the subject matter of Example 54 includes, wherein to modulate the carrier wave with the biological or natural frequency stimulation signal comprises to modulate an amplitude of the carrier wave with the biological or natural frequency stimulation signal.

Example 56 is a method to induce alpha-wave activity in a patient, comprising: providing a modulated stimulation signal using a signal generation circuit, the modulated stimulation signal including a carrier wave modulated with a biological or natural frequency stimulation signal; and providing the modulated stimulation signal to the patient to affect a mood of the patient, the modulated stimulation signal configured to provide the benefit of the biological or natural frequency stimulation signal and the carrier wave to the patient without the patient perceiving the biological or natural frequency stimulation signal, wherein the carrier wave and the modulated stimulation signal have frequencies corresponding to a range of human perception, and wherein the biological or natural frequency stimulation signal comprises a stimulation signal having a frequency greater than 100 Hz and less than 500 kHz, corresponding to a range of frequency where visual changes are not human perceptible.

In Example 57, the subject matter of Example 56 includes, wherein the biological or natural frequency stimulation signal has a frequency greater than 400 Hz and less than 500 kHz.

In Example 58, the subject matter of Examples 56-57 includes, wherein the biological or natural frequency stimulation signal has a frequency of 528 Hz.

In Example 59, the subject matter of Examples 56-58 includes, modulating the carrier wave with the biological or natural frequency stimulation signal using the signal generation circuit.

In Example 60, the subject matter of Example 59 includes, wherein modulating the carrier wave with the biological or natural frequency stimulation signal includes modulating an amplitude of the carrier wave with the biological or natural frequency stimulation signal.

Example 61 is a system to induce alpha-wave activity in a patient, comprising: a signal generation circuit configured to provide a modulated stimulation signal, the modulated stimulation signal including a carrier wave having a carrier frequency outside a range of human perception modulated with a biological or natural frequency stimulation signal comprising a signal having a frequency below 400 Hz corresponding to a range of frequencies where visual changes are human perceptible; and a visual source configured to provide the modulated stimulation signal to the patient to affect a mood of the patient, the modulated stimulation signal configured to provide the benefit of the biological or natural frequency stimulation signal to the patient without the patient perceiving the biological or natural frequency stimulation signal.

In Example 62, the subject matter of Example 61 includes, wherein the signal generation circuit is configured to modulate an amplitude of the carrier wave with the biological or natural frequency stimulation signal.

In Example 63, the subject matter of Examples 61-62 includes, wherein the biological or natural frequency stimulation signal comprises a signal having a frequency below 90 Hz corresponding to a range of frequencies where visual changes are human perceptible.

In Example 64, the subject matter of Examples 61-63 includes, wherein the biological or natural frequency stimulation signal comprises a signal having a frequency below 60 Hz corresponding to a range of frequencies where visual changes are human perceptible.

In Example 65, the subject matter of Examples 61-64 includes, wherein the biological or natural frequency stimulation signal, once modulated onto the carrier wave, is not perceptible to the patient, wherein not perceptible comprises outside a range of human perception.

Example 66 is a method to induce alpha-wave activity in a patient, comprising: providing a modulated stimulation signal using a signal generation circuit, the modulated stimulation signal including a carrier wave having a carrier frequency outside a range of human perception modulated with a biological or natural frequency stimulation signal comprising a signal having a frequency below 400 Hz corresponding to a range of frequencies where visual changes are human perceptible; and providing the modulated stimulation signal to the patient using a visual source to affect a mood of the patient, the modulated stimulation signal configured to provide the benefit of the biological or natural frequency stimulation signal to the patient without the patient perceiving the biological or natural frequency stimulation signal.

In Example 67, the subject matter of Example 66 includes, modulating an amplitude of the carrier wave with the biological or natural frequency stimulation signal using the signal generation circuit.

In Example 68, the subject matter of Examples 66-67 includes, wherein the biological or natural frequency stimulation signal comprises a signal having a frequency below 90 Hz corresponding to a range of frequencies where visual changes are human perceptible.

In Example 69, the subject matter of Examples 66-68 includes, wherein the biological or natural frequency stimulation signal comprises a signal having a frequency below 60 Hz corresponding to a range of frequencies where visual changes are human perceptible.

Example 70 is a system to induce alpha-wave activity in a patient, comprising: a signal generation circuit configured to provide a modulated stimulation signal, the modulated stimulation signal including a carrier wave having a carrier frequency between 20 Hz and 20 kHz modulated with a biological or natural frequency stimulation signal according to a first modulation signal; and an audio source configured to provide the modulated stimulation signal to affect a mood of the patient, the modulated stimulation signal configured to provide the benefit of the biological or natural frequency stimulation signal and the carrier wave to the patient without the patient perceiving the biological or natural frequency stimulation signal, wherein the first modulation signal comprises an alpha-wave signal having a frequency between 7 Hz and 14 Hz.

In Example 71, the subject matter of Example 70 includes, wherein the audio source comprises a speaker and wherein the carrier wave comprises an acoustic signal.

In Example 72, the subject matter of Examples 70-71 includes, wherein the signal generation circuit is configured to modulate an amplitude of the carrier wave with the biological or natural frequency stimulation signal.

Example 73 is a method to induce alpha-wave activity in a patient, comprising: providing a modulated stimulation signal using a signal generation circuit, the modulated stimulation signal including a carrier wave having a carrier frequency between 20 Hz and 20 kHz modulated with a biological or natural frequency stimulation signal according to a first modulation signal; and providing the modulated stimulation signal to the patient using an audio source to affect a mood of the patient, the modulated stimulation signal configured to provide the benefit of the biological or natural frequency stimulation signal and the carrier wave to the patient without the patient perceiving the biological or natural frequency stimulation signal, wherein the first modulation signal comprises an alpha-wave signal having a frequency between 7 Hz and 14 Hz.

In Example 74, the subject matter of Example 73 includes, wherein the audio source comprises a speaker and wherein the carrier wave comprises an acoustic signal.

In Example 75, the subject matter of Examples 73-74 includes, modulating an amplitude of the carrier wave with the biological or natural frequency stimulation signal using the signal generation circuit.

Example 76 is a system to induce alpha-wave activity in a patient, comprising: a signal generation circuit configured to provide a modulated stimulation signal, the modulated stimulation signal including a carrier wave modulated with a biological or natural frequency stimulation signal; and an audio source configured to provide the modulated stimulation signal to affect a mood of the patient, wherein the carrier wave has a frequency outside a range of human hearing, and wherein the modulated stimulation signal is configured to provide the benefit of the biological or natural frequency stimulation signal to the patient without the patient perceiving the biological or natural frequency stimulation signal.

In Example 77, the subject matter of Example 76 includes, wherein the audio source comprises a speaker and wherein the carrier wave comprises an acoustic signal.

In Example 78, the subject matter of Examples 76-77 includes, wherein the biological or natural frequency stimulation signal comprises a signal having at least one of: a frequency between 110 Hz and 112 Hz; a frequency between 7 Hz and 14 Hz; or a frequency of 528 Hz.

In Example 79, the subject matter of Examples 76-78 includes, wherein the signal generation circuit is configured to modulate an amplitude of the carrier wave with the biological or natural frequency stimulation signal.

Example 80 is a method to induce alpha-wave activity in a patient, comprising: providing a modulated stimulation signal using a signal generation circuit, the modulated stimulation signal including a carrier wave modulated with a biological or natural frequency stimulation signal; and providing the modulated stimulation signal to the patient using an audio source to affect a mood of the patient, wherein the carrier wave has a frequency outside a range of human hearing, and wherein the modulated stimulation signal is configured to provide the benefit of the biological or natural frequency stimulation signal to the patient without the patient perceiving the biological or natural frequency stimulation signal.

In Example 81, the subject matter of Example 80 includes, wherein the audio source comprises a speaker and wherein the carrier wave comprises an acoustic signal.

In Example 82, the subject matter of Examples 80-81 includes, wherein the biological or natural frequency stimulation signal comprises a signal having at least one of: a frequency between 110 Hz and 112 Hz; a frequency between 7 Hz and 14 Hz; or a frequency of 528 Hz.

In Example 83, the subject matter of Examples 80-82 includes, modulating an amplitude of the carrier wave with the biological or natural frequency stimulation signal using the signal generation circuit.

Example 84 is a system comprising: a signal generation circuit configured to provide a modulated stimulation signal, the modulated stimulation signal including a carrier wave modulated with a biological or natural frequency stimulation signal; a therapy source configured to provide the modulated stimulation signal to a patient to affect a mood of the patient; and a range sensor configured to determine a range of the patient from the therapy source, and to adjust the modulated stimulation signal using the determined range, wherein the modulated stimulation signal is configured to provide the benefit of the biological or natural frequency stimulation signal to the patient without the patient perceiving the biological or natural frequency stimulation signal.

In Example 85, the subject matter of Example 84 includes, wherein the system is configured to induce neurological activity in the patient, the neurological activity comprising one or more of alpha wave activity, delta wave activity, theta wave activity, beta wave activity, or gamma wave activity in the patient, wherein the biological or natural frequency comprises an alpha wave frequency, delta wave frequency, theta wave frequency, beta wave frequency, or gamma wave frequency, respectively.

In Example 86, the subject matter of Examples 84-85 includes, wherein the range sensor comprises at least one of: a sonar sensor; a radar sensor; a lidar sensor; an image sensor; or a sound sensor.

In Example 87, the subject matter of Examples 84-86 includes, wherein the range sensor is configured to adjust an amplitude of the modulated stimulation signal using the determined range.

In Example 88, the subject matter of Examples 84-87 includes, wherein the range sensor is configured to increase an amplitude of the modulated stimulation signal when the determined range of the patient increases and to decrease the amplitude of the modulated stimulation signal when the determined range of the patient decreases.

In Example 89, the subject matter of Examples 84-88 includes, wherein the range sensor is configured to determine a position of the patient, and to control the therapy source using the determined position of the patient and the determined range of the patient.

In Example 90, the subject matter of Example 89 includes, wherein the range sensor is configured to determine a relative angular displacement of the patient with respect to the therapy source, and to control the therapy source using the determined relative angular displacement of the patient and the determined range of the patient.

Example 91 is a method comprising: providing a modulated stimulation signal using a signal generation circuit, the modulated stimulation signal including a carrier wave modulated with a biological or natural frequency stimulation signal; determining a range of a patient from a therapy source using a range sensor; adjusting the modulated stimulation signal using the determined range; and providing the modulated stimulation signal to the patient using the therapy source to affect a mood of the patient, wherein the modulated stimulation signal is configured to provide the benefit of the biological or natural frequency stimulation signal to the patient without the patient perceiving the biological or natural frequency stimulation signal.

In Example 92, the subject matter of Example 91 includes, wherein providing the modulated stimulation signal comprises to induce neurological activity in the patient, the neurological activity comprising one or more of alpha wave activity, delta wave activity, theta wave activity, beta wave activity, or gamma wave activity in the patient, wherein the biological or natural frequency comprises an alpha wave frequency, delta wave frequency, theta wave frequency, beta wave frequency, or gamma wave frequency, respectively.

In Example 93, the subject matter of Examples 91-92 includes, wherein the range sensor comprises at least one of: a sonar sensor; a radar sensor; a lidar sensor; an image sensor; or a sound sensor.

In Example 94, the subject matter of Examples 91-93 includes, wherein adjusting the modulated stimulation signal using the determined range comprises adjusting an amplitude of the modulated stimulation signal using the determined range.

In Example 95, the subject matter of Examples 91-94 includes, wherein adjusting the modulated stimulation signal comprises increasing an amplitude of the modulated stimulation signal when the determined range of the patient increases and decreasing the amplitude of the stimulation signal when the determined range of the patient decreases.

In Example 96, the subject matter of Examples 91-95 includes, determining a position of the patient using the range sensor; and controlling the therapy source using the determined position of the patient and the determined range of the patient.

In Example 97, the subject matter of Example 96 includes, wherein determining the position of the patient comprises determining a relative angular displacement of the patient with respect to the therapy source, and wherein controlling the therapy source comprises using the determined relative angular displacement of the patient and the determined range of the patient.

Example 98 is a system comprising: a signal generation circuit configured to provide a modulated stimulation signal, the modulated stimulation signal including a carrier wave modulated with a biological or natural frequency stimulation signal; and a therapy source configured to provide the modulated stimulation signal to a patient, the modulated stimulation signal configured to provide the benefit of the biological or natural frequency stimulation signal to the patient without the patient perceiving the biological or natural frequency stimulation signal.

In Example 99, the subject matter of Example 98 includes, wherein the system is configured to induce neurological activity in the patient, and wherein the therapy source is configured to provide the modulated stimulation signal to the patient to induce sleepiness or a relaxed state of the patient.

In Example 100, the subject matter of Example 99 includes, wherein the neurological activity comprises one or more of delta wave activity, theta wave activity, beta wave activity, or gamma wave activity in the patient, wherein the biological or natural frequency comprises a delta wave frequency, theta wave frequency, beta wave frequency, or gamma wave frequency, respectively.

In Example 101, the subject matter of Examples 98-100 includes, wherein the system is configured to induce delta wave activity in the patient, and wherein the therapy source is configured to provide the modulated stimulation signal to the patient to induce sleepiness or improve sleep quality of the patient.

Example 102 is a method comprising: providing a modulated stimulation signal using a signal generation circuit, the modulated stimulation signal including a carrier wave modulated with a biological or natural frequency stimulation signal; and providing the modulated stimulation signal to a patient using a therapy source, providing the benefit of the biological or natural frequency stimulation signal to the patient without the patient perceiving the biological or natural frequency stimulation signal.

In Example 103, the subject matter of Example 102 includes, wherein providing the modulated stimulation signal comprises to induce neurological activity in the patient to induce sleepiness or a relaxed state in the patient.

In Example 104, the subject matter of Example 103 includes, wherein the neurological activity comprises one or more of delta wave activity, theta wave activity, beta wave activity, or gamma wave activity in the patient, wherein the biological or natural frequency comprises a delta wave frequency, theta wave frequency, beta wave frequency, or gamma wave frequency, respectively.

In Example 105, the subject matter of Examples 102-104 includes, wherein providing the modulated stimulation signal comprises to induce delta wave activity in the patient to induce sleepiness or improve sleep quality of the patient.

Example 106 is at least one machine-readable medium including instructions that, when executed by processing circuitry, cause the processing circuitry to perform operations comprising: generating a modulated stimulation signal, the modulated stimulation signal including a carrier wave modulated with a biological or natural frequency stimulation signal across a first frequency range of a first modulation signal according to a second modulation signal; and providing the modulated stimulation signal to a patient to provide the benefit of the biological or natural frequency stimulation signal to the patient without the patient perceiving the biological or natural frequency stimulation signal.

In Example 107, the subject matter of Example 106 includes, wherein providing the modulated stimulation signal to the patient using a therapy source comprises using one of an audio, visual, or electromagnetic source configured to provide the modulated stimulation signal to the patient.

In Example 108, the subject matter of Examples 106-107 includes, wherein the carrier wave is imperceptible to the patient, and wherein the biological or natural frequency stimulation signal, once modulated onto the carrier wave, is not perceptible to the patient, wherein not perceptible comprises outside a range of human perception.

In Example 109, the subject matter of Examples 106-108 includes, wherein the carrier wave has a frequency corresponding to a range of human perception, and wherein the biological or natural frequency stimulation signal, once modulated onto the carrier wave, is not perceptible to the patient.

In Example 110, the subject matter of Examples 106-109 includes, wherein the modulated stimulation signal comprises the carrier wave amplitude modulated across the first frequency range at a frequency of the second modulation signal.

In Example 111, the subject matter of Examples 106-110 includes, wherein the second modulation signal has a second frequency range, and wherein the modulated stimulation signal comprises the carrier wave amplitude modulated across the first frequency range by the second frequency range.

In Example 112, the subject matter of Example 111 includes, wherein the first and second frequency ranges of the first and second modulation signals, when provided by the therapy source, correspond to ranges of human perception, and wherein the first and second frequency ranges of the first and second modulation signals, once modulated onto the carrier wave, are not perceptible to the patient.

In Example 113, the subject matter of Examples 111-112 includes, wherein the first frequency range of the first modulation signal is between 110 Hz and 112 Hz, and wherein the second frequency range of the second modulation signal is between 7 Hz and 14 Hz.

In Example 114, the subject matter of Examples 106-113 includes, wherein the operations further comprise: determining a range of the patient from the therapy source using information from a range sensor; and adjusting the modulated stimulation signal using the determined range.

In Example 115, the subject matter of Examples 106-114 includes, wherein the operations further comprise: modulating the carrier wave with the biological or natural frequency stimulation signal using the signal generation circuit, including modulating the carrier wave across the first frequency range of the first modulation signal according to the second modulation signal.

Example 116 is at least one machine-readable medium including instructions that, when executed by processing circuitry, cause the processing circuitry to perform operations comprising: generating a modulated stimulation signal, the modulated stimulation signal including a carrier wave modulated with a biological or natural frequency stimulation signal; and providing the modulated stimulation signal to the patient, providing the benefit of the biological or natural frequency stimulation signal to a patient without the patient perceiving the biological or natural frequency stimulation signal.

In Example 117, the subject matter of Example 116 includes, wherein providing the modulated stimulation signal comprises to induce neurological activity in the patient to induce sleepiness or a relaxed state in the patient.

In Example 118, the subject matter of Example 117 includes, wherein the neurological activity comprises one or more of delta wave activity, theta wave activity, beta wave activity, or gamma wave activity in the patient, wherein the biological or natural frequency comprises a delta wave frequency, theta wave frequency, beta wave frequency, or gamma wave frequency, respectively.

In Example 119, the subject matter of Examples 116-118 includes, wherein providing the modulated stimulation signal comprises to induce delta wave activity in the patient to induce sleepiness or improve sleep quality of the patient.

In Example 120, the subject matter of Examples 98-119 optionally include a signal generation circuit configured to provide a modulated stimulation signal, the modulated stimulation signal including a carrier wave modulated with a biological or natural frequency stimulation signal; and a therapy source configured to provide the modulated stimulation signal to a patient, the modulated stimulation signal configured to provide the benefit of the biological or natural frequency stimulation signal to the patient without the patient perceiving the biological or natural frequency stimulation signal.

In Example 121, the subject matter of Examples 98-120 optionally includes wherein the electromagnetic source comprises a directional antenna.

In Example 122, the subject matter of Examples 98-121 optionally includes wherein the system comprises a therapy device comprising the signal generation circuit and the patch antenna, wherein the patch antenna is printed on a printed circuit board (PCB) configured to be located within the therapy device, herein the patch antenna has a front-to-back power ratio of at least 10.

In Example 122, the subject matter of Examples 98-121 optionally includes, wherein the patch antenna has a field of view, when the therapy device is configured for use, that is at least 90 degrees.

In Example 123, the subject matter of Examples 98-122 optionally includes wherein the signal generation circuit is configured to control a transmit power limit of the modulated stimulation signal transmitted by the directional antenna to be below a safety limit published or set by a standard body.

In Example 124, the subject matter of Examples 98-123 optionally includes wherein the carrier wave has a frequency of a published Wi-Fi range, including one or more of 2.4 GHz, 5 GHz, or 5.7-5.8 GHz.

Example 125 is at least one machine-readable medium including instructions that, when executed by processing circuitry, cause the processing circuitry to perform operations to implement of any of Examples 1-124.

Example 126 is an apparatus comprising means to implement of any of Examples 1-124.

Example 127 is a system to implement of any of Examples 1-124.

Example 128 is a method to implement of any of Examples 1-124.

The above detailed description is intended to be illustrative, and not restrictive. The scope of the disclosure should, therefore, be determined with references to the appended claims, along with the full scope of equivalents to which such claims are entitled.

What is claimed is:

1. A system comprising:
a signal generation circuit configured to provide a modulated stimulation signal, the modulated stimulation signal including a carrier wave modulated with a biological or natural frequency stimulation signal; and
a therapy source configured to provide the modulated stimulation signal to a patient, the modulated stimulation signal configured to provide the benefit of the biological or natural frequency stimulation signal to the patient without the patient perceiving the biological or natural frequency stimulation signal.

2. The system of claim 1, wherein the therapy source comprises an electromagnetic source.

3. The system of claim 2, wherein the electromagnetic source comprises a light source.

4. The system of claim 3, wherein the carrier wave comprises a human-perceptible visible light signal.

5. The system of claim 4, wherein the biological or natural frequency stimulation signal comprises a human-imperceptible signal above an upper range of human visual perception, wherein the human-imperceptible signal includes a frequency greater than 90 Hz.

6. The system of claim 5, wherein the human-imperceptible signal includes a frequency greater than 100 Hz.

7. The system of claim 8, wherein the human-imperceptible signal includes a frequency greater than 400 Hz.

8. The system of claim 2, wherein the carrier wave has a frequency corresponding to a range of human perception, and
wherein the biological or natural frequency stimulation signal, once modulated onto the carrier wave, is not perceptible to the patient.

9. The system of claim 2, wherein the carrier wave comprises a human-imperceptible carrier wave.

10. The system of claim 2, wherein the biological or natural frequency stimulation signal comprises a human-perceptible or a human-imperceptible signal separate from the carrier wave.

11. The system of claim 10, wherein the frequency of the human-imperceptible carrier wave is above 100 kHz.

12. The system of claim 10, wherein the human-imperceptible signal includes a frequency greater than 100 Hz.

13. The system of claim 10, wherein the human-perceptible signal includes a frequency lower than 90 Hz.

14. The system of claim 13, wherein the human-perceptible signal includes a frequency lower than 60 Hz.

15. The system of claim 9, wherein the carrier wave is imperceptible to the patient, and
wherein the biological or natural frequency stimulation signal, once modulated onto the carrier wave, is not perceptible to the patient, wherein not perceptible comprises outside a range of human perception.

16. The system of claim 9, wherein the therapy source includes an emitter comprising a coil or a quarter-wave, half-wave, or full-wave antenna.

17. The system of claim 2, comprising:
a memory to store the modulated stimulation signal,
wherein the signal generation circuit is configured to receive the modulated stimulation signal from memory and to provide the modulated stimulation signal to the therapy source.

18. The system of claim 17, wherein the biological or natural frequency stimulation signal comprises an alpha-wave signal recorded from the patient or one or more other individuals.

19. The system of claim 18, wherein the alpha-wave signal comprises an alpha-wave signal of the patient.

20. The system of claim 2, wherein the biological or natural frequency stimulation signal comprises an alpha wave.

21. The system of claim 20, wherein the biological or natural frequency stimulation signal comprises a digital representation of an alpha wave recorded from the patient or one or more other patients,
wherein the signal generation circuit is configured to determine the modulated stimulation signal using the recorded alpha wave.

22. The system of claim 2, wherein the biological or natural frequency stimulation signal comprises a delta wave, a theta wave, a beta wave, or a gamma wave.

23. The system of claim 2, wherein the biological or natural frequency stimulation signal comprises a Solfeggio frequency.

24. The system of claim 2, wherein the modulated stimulation signal is provided using amplitude modulation.
